(19) United States
(12) Patent Application Publication
(10) Pub. No.: US 2012/0108425 A1
(43) Pub. Date: May 3, 2012

(54) BIOSYNTHESIS OF GOLD AND SILVER NANOPARTICLES FOR STABILITY AND EXTENDED SHELF-LIFE OF ANTAGONISTIC ACTIVITIES

(76) Inventors: Balasubramanian Mythili Gnanamangai, JR., Namakkal District (IN); P. Ponmurugan Ponnusamy, SR., Namakkal Dt (IN)

(21) Appl. No.: 12/916,497

(22) Filed: Oct. 30, 2010

(51) Int. Cl. A01N 59/16; C22C 5/02; A01N 63/00; A01P 3/00; A01P 21/00; A01P 1/00; C22C 5/06; C12P 3/00; B82Y 5/00

(52) U.S. Cl. 504/117; 420/501; 420/507; 424/93.1; 504/187; 424/618; 424/649; 435/168; 977/773; 977/915

(57) ABSTRACT

The use of microbial nanoparticles to stabilize antifungal activity is described. The preferred silver and gold nanoparticles are formed from various categories of microbes like bacteria, fungi and actinomycetes. The silver and gold nanoparticles synthesized from microbes, which can efficiently work as biocontrol/biofertilizer agent in field. The stabilized antagonistic activities of the microbes can be used for many other purposes with respect to the microbe selected for nanoparticle synthesis. Here the selected microbes were an indigenous organisms isolated from the tea fields that can control various diseases, individually and in combination with other microbe as biofertilizer.

### FIELD OF THE INVENTION

The present invention relates to extension or stabilization of antagonistic activities using silver and gold nanoparticles synthesized from microbes and, more particularly, from biocontrol agents isolated from tea fields that can be applied to tea plantation as biofertilizers in an aspect of controlling various diseases.

### BACKGROUND ART

The present invention is directed to a new method for stabilizing the antagonistic activities of microbes. The invention is particularly directed to the provision of various types of bioformulations containing stabilized antifungal activity with effectiveness in controlling wide range of diseases. The invention is especially useful in the field of agriculture especially in organic farming where the dependence is concentrated on bioformulation alone.

Any antagonistic activity (antifungal/antibacterial) is inherently unstable in nature due to its susceptibility to decomposition by trace levels of fluctuations in temperature and other environmental factors including time periods. These factors involve in disturbing the nature of the antagonistic activities. That certainly degrades the quality of the bioformulations used mainly for its antagonistic properties.

In order to address this stability problem, it is necessary to prepare or cultivate a microbe possessing antifungal activity that has extended shelf-life of exhibiting antagonistic property. Nanoparticles such as gold and silver are stable carriers and intrinsically possess the nature of antimicrobial activity. Nanoparticles are viewed as the fundamental building blocks of nanotechnology [G. A. Mansoori, Principles of Nanotechnology Molecular-Based Study of Condensed Matter in Small Systems, World Scientific Pub. Co., Hackensack, N.J., (2005)].

Nanoparticles themselves have useful applications not only in areas such as medicine and molecular biology research but also in the field of agriculture and food processing. In particular, silver and gold nanoparticles may be used as antimicrobial agents against bacteria, viruses, and fungi, including drug-resistant strains of microorganisms. Typically, bacteria have diameters in the micron range, while viruses have diameters less than a micron in size. The fact that the silver nanoparticles are so small allows them to interact readily with such microorganisms. The antimicrobial action occurs because the silver nanoparticles interfere with the enzymatic metabolism of oxygen by the microbes, which effectively “suffocates” and kills the particular microorganism. The nanoscale size of silver nanoparticles means that the particles have a very large surface area, therefore only a small volume of silver nanoparticles is required to act as an effective antagonistic agent.

Silver and gold nanoparticles are usually produced on a small laboratory scale using methods such as chemical vapor deposition, irradiation or chemical reduction of metal salts. However, the eco-friendly nanoparticles that do not produce toxic wastes in their process synthesis protocol will be the only remedy when subjecting nanoparticles to agriculture purpose. To achieve this, scientists in the field of synthesis and assembly of nanoparticles are inclined to shift to benign synthesis processes, which happen to be mostly of a biological nature [G. A. Mansoori, Principles of Nanotechnology Molecular-Based Study of Condensed Matter in Small Systems, World Scientific Pub. Co., Hackensack, N.J., (2005)].

In recent times, the utilization of biological entities has emerged as a novel method for the synthesis of nanoparticles. Biotechnology approaches toward the synthesis of nanoparticles can have many advantages, such as a greater ease with which the process can be scaled up, economic viability, possibility of readily covering large surface areas by suitable growth of the microbes, which is considered to be the major advantage in the field of agriculture for easier production of biofertilizers.

The use of nanoparticles for antimicrobial activity was described in U.S. patent Ser. No. 11/562,554

Tea is a perennial woody plant having a single main stem from which numerous branches are developed to a crown of leaves to get a bushy appearance. Being a monoculture crop, it provides a stable microclimate for a number of pests and diseases. A large number of pathogenic organisms in different parts of the plants are available in the ecological niche. The crop loss due to pests and diseases is around 10-15%. The majority of diseases in tea are of fungal origin and one each of virus and algae. In a recent monograph on tea diseases nearly 400 organisms are involved. Irrespective of the pathogen and the parts affected, the disease symptoms manifest as debilitation and sometimes death of bushes.

Among the tea diseases, Leaf diseases are important due to the obvious reason that tea plants are cultivated for its young succulent leaves to manufacture tea dhool. These diseases affect the crop by their indirect effect on bush health, but if both young and mature leaves are attacked, the quantity of harvest is reduced. Leaf blight, leaf spot, leaf rot and leaf rust are the common leaf diseases of the tea bush. Among these bird's eye spot disease caused by Cercospora theae Petch is more serious in tea plantations.

Cercospora theae is a pathogenic fungus, which belongs to the family Deutromycetes of the Division Mycota. It occurs in almost all tea growing areas of the world. It has been reported that the disease is more common in mature tea plantations in India, Bangladesh, Sri Lanka, China, Japan and Kenya. Infection occurs both on the leaves and young shoots of the plant. The disease affected leaves appears with small black, round, sunken necrotic spots with a red colored margin.

Though loss in terms of yield due to this disease has not been estimated, it definitely affects the growth of plants in their early stage of development. Providing proper shade for tea plants, proper drainage, soil aeration and proper manuring especially with potassium can control the disease. Foliar spraying of copper fungicides to bird's eye spot affected plants has been found useful for controlling the disease to some extent. However, there is no satisfactory control recorded due to the long and indefinite life cycle of the pathogen.

Although leaf spot disease of tea is known for over 40 years in India, the disease has become a major problem only in recent years on the estates now resort to large scale replanting, infilling and inter row planting to increase production and productivity. Unfortunately, majority of the clones and seedlings used for this are susceptible to the disease. Further, the change in climatic condition aggravates the situation.

In phytopathological studies, distribution of the disease, its host ranges, and varietal resistance are important parameters in determining its economic importance. In South India, more than 45 clones and five seed stocks are being available. Similarly in northeast India also, more than 50 clones and 100 seedlings are available to tea planters towards not only in getting more yields but also resistant to pests, diseases, frost and drought. Among the clones and jats, most of them are susceptible to this bird's eye spot disease.

### OBJECTIVE OF THE INVENTION

The basic objective of the present invention is to provide a new reliable methodology to use nanoparticles for stabilizing the antimimcrobial properties of the biocontrol agents.

Another objective of the present invention is to extend the shelf-life of biocontrol agents and its efficacy of biocontrol activity.

Yet another objective of the invention is directed to longterm usage of bioformulation for its efficiency in an affordable cost

Further objective of the invention is to obtain one organism from each category of microbes (bacteria, fungi and actinomycetes) that can synthesis both gold and silver nanoparticles.

After nanoparticle synthesis, the nature and efficacy of the microbes to control the disease was studied in detail at various time intervals for a period of one year for a foliar disease in tea plantations caused by Cercospora theae.

### SUMMARY OF THE INVENTION

The present invention is directed to a new method for stabilizing or extension of shelf-life of microbes having antimicrobial properties. The method is based on the discovery that nanoparticles conjugated antibiotics were more efficient. Moreover, nanoparticles were shown to take up by phagocytic cells and held promises as carrier for the treatment of intracellular infections with several antibiotics. This interaction is believed to effectively destroy pathogens in the fields when optimal organism was selected for biocontrol activity.

The stabilization method of the present invention has several advantages over prior methods. For example, the nanoparticles stabilize the antimicrobial properties for certain longer period. In contrast, the use of microbes or microbial consortia having antagonistic activity for plant growth promotion and biocidal activity has less survival period and are non-stable in nature. The nano-particles also have positive effects apart from the stabilization of antimicrobial activities, such as enhanced antagonistic activity when synthesized by the same biocontrol agent.

According to the basic aspect of the present invention a new methodology has been standardized to control the plant pathogens especially like Cercospora theae isolates from ‘leaf spot’ infected tea leaves collected from different agroclimatic zones such as, Koppa of Karnataka, Munnar of Kerala and Valparai of Tamil Nadu in southern India. The bird's eye spot infected tea leaves have been collected from South Indian tea plantations. The following isolation work has been performed under invitro conditions. Three isolates of C. theae were subjected to the proposed study of controlling the disease using nanoparticles. Each one from three category of microbes (actinomycetes, fungi, bacteria) were isolated from tea plants (endophytes) so as to use an most efficient biocontrol agent from each group of microbe.

The endophytes were evaluated individually against the plant pathogen and were then subjected for field trials to enhance the plant growth. Similarly, the endophytes were identified and subjected to analysis of capability to synthesis gold and silver nanoparticles. The three endophytes were identified as Streptomyces spp. of actinomycetes, Trichoderma atroviride of fungi and Pseudomonas aeruginosa of bacteria. Both the biomass and the culture filtrate of the above mentioned microbes were subjected to gold and silver nanoparticles synthesis. The synthesized nanoparticles were analysed with uv spectrophotometer, AFM, FITR and particle size analyzer. The filtrates after synthesis of nanoparticles were subjected to antifungal activity and compared with the filtrates of the organisms with their respective controls.

The filtrate was added in 10% concentration to the solid medium before solidification and was then inoculated with the plant pathogen the growth was recorded consecutively for 14 days. It was compared with controls prepared without filtrates and with filtrates uninoculated with biocontrol agent. The percentage inhibition was calculated and compared without synthesis of nanoparticles.

The same evaluation study was carried at every two month intervals for a period of one year by preserving all the filtrates in less than 4° C. accompanied by without preservation to check the viability of the antimicrobial compound in room temperatures after and before nanoparticle synthesis.

### EXAMPLES

The present invention is further illustrated by the following specific examples.

### Example 1

### Isolation of Endophytes (Bacteria, Fungi and Actinomycetes)

In this example, various healthy tea leaves collected from different zones of South India were used for experimentation. In order to avoid the ephiphytic fungi surface sterilization method was followed, where thirty (5 mm diam) leaf disks of each clones were prepared using flame sterilized cork borer and transferred separately and submerged in 70% ethanol for 1 minutes, and again kept into 70% ethanol for 1 minute.

Thereafter the disks were serially washed in ten changes of sterile distilled water, and leaves were inoculated of each clone on six petridishes (5 leaf disc/plate) of which three were holding mature leaves and three plates with young leaves over nutrient agar, Czapek-Dox agar and Casein Nitrate agar and incubated at 25±1° C. under fluorescent light for 7 days. Isolated bacterial, fungal and actinomycetes colonies were subcutured and maintained separately for identification. All the organisms isolated were subjected to standard morphological and biochemical characterization and were identified as Pseudomonas aeuroginosa, Trichoderma atroviride and Streptomyces spp. respectively.

### Example 2

### Preparation of Endophytes

The endophytes were prepared at 28° C. in petriplates, containing Kings B media, Potato Dextrose Agar (PDA) media and Casein Nitrate Agar media which were specific microbiological media for culturing the above mentioned microbes in respective order. For the synthesis of nano-particles, the microbes was grown in 200 mL bottles each con-taining 100 mL of a liquid medium. The bottles were stored at a temperature between 25-28° C., with continuous mixing by a magnetic stirrer (rotary shaker, Orbitech-LE-IL, Scigenics, India) at 150 rpm for 72 hours.

The biomass was then separated from the culture broth by sterile filter paper and the settled mycelia were washed three times with sterile distilled water. In case of bacteria and actinomycetes the broth was centrifuged at 5000 rpm for 5 min and the pellet was washed with sterile distilled water three times and was used as biomass for the synthesis of silver and gold nanoparticles.

### Example 3

### Biosynthesis of Silver and Gold Nanoparticles

In a typical biosynthesis production scheme of silver and nanoparticles according to the present invention, 10 g of each wet microbial biomass fungus was mixed with a 100 ml aqueous solution of 1 mM silver nitrate (AgNO₃) and chloroauric acid (HAuCl₄.XH₂O). The mixture was placed at 100 rpm rotating shaker at 28° C. for 120 hours. In this process, silver and gold nanoparticles were produced through reduction of the silver ions to metallic silver and auric ions to metallic gold by enzymes produced by the microbial biomass.

### Example 4

### Analysis of Nanoparticles

Optical spectroscopy has been widely used for the characterization of nanomaterials. In the examples described herein, three different spectroscopy techniques were used to fully characterize the silver and gold nanoparticles produced. They include absorption UV-Visible light spectroscopy, AFM, FITR and particle size analyzer. UV-Visible light spectroscopy was used to follow up with the reaction process. The reduction of silver and gold ions was routinely monitored by visual inspection of the solution as well as by measuring the UV-Visible spectra of the solution by periodic sampling of 2 mL aliquots of the aqueous component. The UV-Visible spectroscopic measurements were recorded on a U-2900E, Model no. 2.1′-0003, UV-visible spectrophotometer, Hitachi, Tokyo, Japan.

### Example 5

### Comparative Analysis of Antimicrobial Activity

The culture filtrates of endophytes were collected and tested for biocontrol activity especially against the plant pathogen C. theae. The filtrate was added at 10% concentration to the PDA before solidification and inoculated with the phytopathogen C. theae. The radial growth was measured at regular intervals and percent inhibition was calculated using the formula P=(^{A}R_{C}−^{A}R_{T})/R_{C}*100 where P is the percentage growth inhibition, ^{A}R_{C} is the average radial growth of the pathogen in control plates and ^{A}R_{T} is the average radial growth of the pathogen in treated plates.

Similarly, the culture filtrate obtained after silver and gold nanoparticles were subjected to the same pathogen and percent inhibition was calculated.

TABLE 1
Initial Effect of biosynthesized nanoparticles against C. theae

| Isolates | Endophytes or | Radial growth of C. theae on 14^{th} day in mm | | | | | |
|---|---|---|---|---|---|---|---|
| of | Biocontrol | Culture filtrate before | | Culture filtrate after silver | | Culture filtrate after gold | |
| C. theae | agent used | nanoparticle synthesis | | nanoparticle synthesis | | nanoparticle synthesis | |
| KC10 | Streptomyces spp. | 50.7 | (40.6) | 20.7 | (66.3) | 30.3 | (61.9) |
| | Trichoderma atroviride | 53.3 | (37.5) | 34.7 | (62.2) | 36.0 | (55.9) |
| | Pseudomonas aeuroginosa | 59.7 | (30.1) | 24.3 | (57.8) | 38.0 | (38.1) |
| MC24 | Streptomyces spp. | 62.7 | (25.4) | 22.0 | (67.9) | 27.0 | (68.1) |
| | Trichoderma atroviride | 62.0 | (26.2) | 22.0 | (75.2) | 22.0 | (75.2) |
| | Pseudomonas aeuroginosa | 65.3 | (22.2) | 21.0 | (61.9) | 32.0 | (56.9) |
| VC38 | Streptomyces spp. | 54.7 | (34.9) | 22.7 | (61.5) | 32.3 | (62.2) |
| | Trichoderma atroviride | 55.3 | (34.1) | 20.0 | (76.2) | 20.0 | (77.8) |
| | Pseudomonas aeuroginosa | 58.3 | (30.6) | 17.0 | (65.6) | 29.3 | (63.5) |

Value in parantheses indicate percentage inhibition of growth of C. theae

The antagonistic activity was exhibited more by the culture filtrates of silver nanoparticles, followed by culture filtrate of gold nanoparticles and culture filtrate of endophytes before nanoparticle synthesis.

### Example 6

### Comparative Analysis of Antimicrobial Activity after Six Month

The above mentioned method was performed after six months of interval with the same culture filtrates that has been stored at 4° C. The percentage inhibition was recorded to determine the stability of antagonistic activity.

The difference in percent inhibition of the pathogen's growth exhibited by the silver nanoparticle culture filtrate and gold nanoparticle culture filtrate in the initial period of harvest and after storage for six month was not significant. Whereas the difference was significant among initial and stored culture filtrate that doesn't undergo nanoparticle synthesis, stating the stability and viability of antagonistic activity was more after nanoparticle synthesis.

TABLE 2
Effect of biosynthesized nanoparticles against C. theae after sixth month

| Isolates | Endophytes or | Radial growth of C. theae on 14^{th} day in mm | | | | | |
|---|---|---|---|---|---|---|---|
| of | Biocontrol | Culture filtrate before | | Culture filtrate after silver | | Culture filtrate after gold | |
| C.theae | agent used | nanoparticle synthesis | | nanoparticle synthesis | | nanoparticle synthesis | |
| KC10 | Streptomyces spp. | 57.0 | (35.2) | 30.3 | (64.3) | 34.3 | (60.9) |
| | Trichoderma atroviride | 58.3 | (33.7) | 34.0 | (60.2) | 39.7 | (53.9) |
| | Pseudomonas aeuroginosa | 63.7 | (27.6) | 38.0 | (54.8) | 34.7 | (36.1) |
| MC24 | Streptomyces spp. | 63.3 | (26.4) | 27.0 | (65.9) | 28.7 | (65.1) |
| | Trichoderma atroviride | 64.0 | (25.6) | 22.0 | (73.2) | 22.0 | (73.2) |
| | Pseudomonas aeuroginosa | 69.7 | (19.0) | 32.0 | (60.9) | 27.3 | (54.9) |
| VC38 | Streptomyces spp. | 76.7 | (10.5) | 32.3 | (60.5) | 34.0 | (60.2) |
| | Trichoderma atroviride | 77.3 | (9.7) | 20.0 | (73.8) | 20.0 | (73.2) |
| | Pseudomonas aeuroginosa | 79.0 | (7.8) | 29.3 | (62.6) | 28.0 | (61.5) |

Value in parantheses indicate percentage inhibition of growth of C. theae

### Example 7

### Comparative Analysis of Antimicrobial Activity after One Year

The above mentioned method was performed after one year of interval with the same culture filtrates that has been stored at 4° C. The percentage inhibition was recorded to determine the stability of antagonistic activity. Negligible amount of antifungal activity was determined in culture filtrate that doesn't undergo nanoparticle synthesis whereas the filtrate underwent silver and gold nanoparticles exhibited antifungal activity in a significant level.

The difference in percent inhibition of the pathogen's growth exhibited by the silver and gold nanoparticle culture filtrate in the initial period of harvest and after storage for six months with stored nano filtrate of one year was not significant. Similarly, the difference was significant among initial and six month stored culture filtrate when compared with one year stored culture filtrate that doesn't undergo nanoparticle synthesis, which clearly indicates that the stability and viability of antagonistic activity was maintained even after a period of one year. On comparing among the percent inhibition revealed by culture filtrate that underwent nanoparticle synthesis and that doesn't undergo nano synthesis, the antagonistic property was almost lost by one year stored culture filtrate that that doesn't undergo nanoparticle synthesis. The overall results clearly states the stability and antagonistic activity was more in silver nanosynthesized culture filtrate, which may be due to indigenous antimicrobial and stabilization property procured by silver nanoparticles [Virender K. et al, Silver nanoparticles: Green synthesis and their antimicrobial activities, Advances in Colloid and Interface Science, 2009, 83-96, 145]. This is then followed by, the culture filtrate subjected to gold nanoparticle synthesis which may be due to the efficient conjugational activity of antagonistic compounds alone [Saha et al, In Vitro Structural and Functional Evaluation of Gold Nanoparticles Conjugated Antibiotics, Nanoscale Res Lett, 2007, 614-622, 2]. As the indigenous culture filtrate that doesn't undergo any nanoparticle synthesis was unstable after one year of storage their efficacy as biocontrol agent in formulation was poor.

TABLE 3
Effect of biosynthesized nanoparticles against C. theae after one year.

| Isolates | Endophytes or | Radial growth of C. theae on 14^{th} day in mm | | | | | |
|---|---|---|---|---|---|---|---|
| of | Biocontrol | Culture filtrate before | | Culture filtrate after silver | | Culture filtrate after gold | |
| C. theae | agent used | nanoparticle synthesis | | nanoparticle synthesis | | nanoparticle synthesis | |
| KC10 | Streptomyces spp. | 85.3 | (5.2) | 34.3 | (59.2) | 41.0 | (52.0) |
| | Trichoderma atroviride | 86.0 | (4.4) | 39.7 | (21.7) | 41.7 | (53.7) |
| | Pseudomonas aeuroginosa | 86.7 | (3.7) | 34.7 | (20.7) | 41.0 | (27.6) |
| MC24 | Streptomyces spp. | 84.0 | (6.7) | 28.7 | (64.9) | 35.0 | (57.9) |
| | Trichoderma atroviride | 85.3 | (5.2) | 22.0 | (64.9) | 35.3 | (60.7) |
| | Pseudomonas aeuroginosa | 86.3 | (4.1) | 27.3 | (50.4) | 35.3 | (43.6) |
| VC38 | Streptomyces spp. | 84.0 | (6.7) | 34.0 | (58.5) | 44.3 | (47.2) |
| | Trichoderma atroviride | 86.3 | (4.1) | 20.0 | (63.4) | 47.3 | (47.4) |
| | Pseudomonas aeuroginosa | 87.7 | (2.6) | 28.0 | (70.5) | 34.3 | (37.2) |

Value in parantheses indicate percentage inhibition of growth of C. theae

What is claimed is:

1. Use of nanoparticles for stabilizing the antagonistic properties and preparing bioformulation in application into the agriculture field.

2. A method for making highly effective antimicrobial bioformulation amended with nanoparticles, as in claim 1, comprises the steps of:
i. obtaining a talc preparation made from the microbes or effective biocontrol agent grown in broth and filtrate containing self synthesized nanoparticles mixed talc in any ratio.
ii. Where microbial nanoparticles synthesized from the indigenous strain especially an endophytes belonging to any category such as bacteria, fungi and actinomycetes.
iii. The nanoparticles either synthesized from culture filtrate or from biomass of the indigenous biocontrol agent.

3. Using the method of claim 2, the microbes belonging to the group of bacteria, fungi or actinomycetes, which is an endophyte isolated from tea leaves used for synthesis of gold and silver nanoparticles especially from any bacteria in the group of Pseudomonas aeuroginosa, any fungi in the group of Trichoderma atroviride and any actinomycetes under the genus Streptomyces spp., were claimed.

4. The microbial silver or gold nanoparticle as in claim 1, for the antagonistic properties against any plant pathogens is claimed.

5. The use silver and gold nanoparticles for fungicide or biocontrol formulation in form of liquid or powder is claimed.

6. The process of conjugation of biosynthesized nanoparticles with the antimicrobial compound for increase in stability is claimed.

7. The process of using any endophytes with conjugated to its own nanoparticles for the prevention or treatment of plant disease was claimed.

8. The use of sliver or gold nanoparticles conjugated with efficient biocontrol agents or plant growth promoting particles for its application in field as plant growth promoters is claimed.
